(19) United States
(12) Patent Application Publication
(10) Pub. No.: US 2021/0186792 A1
(43) Pub. Date: Jun. 24, 2021

(54) METHOD AND SYSTEM FOR MEASURING ERGONOMIC LOAD

(71) Applicant: BIOSERVO TECHNOLOGIES AKTIEBOLAG, Kista (SE)

(72) Inventors: Martin Oskar Gustaf EWALDSSON, Sigtuna (SE); Jan Johan Fredrik SLEMAN, Enskede (SE)

(21) Appl. No.: 17/057,480

(22) PCT Filed: May 17, 2019
(86) PCT No.: PCT/EP2019/062790

§ 371 Date: Nov. 20, 2020

(30) Foreign Application Priority Data
May 25, 2018 (SE) 1850631-1

(51) Int. Cl. A61H 1/02; G16H 40/67; G16H 50/30

(52) U.S. Cl. CPC A61H 1/02; G16H 40/67; G16H 50/30; A61H 1/0237; A61H 1/0281; A61H 2230/605; A61H 1/0292; A61H 2201/165; A61H 2201/5007; A61H 2201/5061; A61H 1/0285

(57) ABSTRACT

Method when a user performs a work task, wherein the user wears and uses a wearable, actively controlled piece of force-assisting equipment (110) used to assist the user in the performing of the said work task, which force-assisting equipment (110) comprises a force-exerting means (112,113) for assisting the user in applying a particular force and/or for performing movements of a particular type, and a sensor means (115) for sensing a force applied by the user, wherein an assisting force exerted by said force-exerting means (112,113) is feedback-controlled based on an instantaneous measurement value from the sensor means (115). The invention is characterised in that the method comprises the steps of during the performance of said work task, measuring said measurement value using the sensor means (115) for use in said feedback control; and using the measurement value to automatically calculate an actual ergonomic risk or load value for the performed work task using the said force-assisting equipment (110). The invention also relates to a system and a computer software product.

The present invention relates to a method and a system for measuring ergonomic load and/or risk. In particular, it is related to such a method using a wearable, actively controlled force-assisting piece of equipment, such as an already existing such piece of equipment.

Conventionally, ergonomic load and risk is evaluated for various work tasks. For instance, evaluation models such as HAL-TLV (Hand Activity Level Threshold Limit Value) and KIM (Key Indicator Method) are used in industry, in which various movement patterns, postures, w forces, as well as user fatigue and effort, are manually assessed and then used to calculate scores and similar values, in turn giving an indication of ergonomic risk for individual work tasks over time.

Such ergonomic risk measurement is very important, since the said scores can be used for early identification and prevention of ergonomic problems; for work force and production planning; and for development of production facilities. However, due to their manual character they are expensive and cumbersome to perform. In addition, the scoring is highly dependent on the measurement conditions, yielding high variation.

Also known is to use wearable, actively controlled, force-assisting equipment when performing various work tasks. For instance, such equipment in the form of different gloves is described in US 20130226350 A1 and WO 2008027002 A.

There are many types of such equipment, including not only gloves, but support gear for arms and/or legs, and even complete exoskeletons, for use in different situations. Common to all of these types of wearable equipment is that they use an active control loop, such as a feedback loop, to assist the wearing user to apply a particular force and/or to perform natural movement, which control loop is arranged to assist or amplify physical activities performed by a user using a force exerted by some type of motor. In many work situations, the user performing the task in question uses such a wearable piece of equipment.

As mentioned above, it would be desirable to be able to assess ergonomic risk in a better, more efficient, way. Also, it would be desirable to be able to plan, monitor and supervise ergonomically risky work tasks in a better way than what is the case today.

The present invention solves these and other problems, and in particular for work tasks that involve the use of a wearable actively controlled force assist equipment of the above described type.

Hence, the invention relates to a method when a user performs a work task, wherein the w user wears and uses a wearable, actively controlled piece of force-assisting equipment used to assist the user in the performing of the said work task, which force-assisting equipment comprises a force-exerting means for assisting the user in applying a particular force and/or for performing movements of a particular type, and a sensor means for sensing a force applied by the user, wherein an assisting force exerted by said force-exerting means is feedback-controlled based on an instantaneous measurement value from the sensor means, which method is characterised in that the method comprises the steps of a) during the performance of said work task, measuring said measurement value using the sensor means for use in said feedback control; and b) using the measurement value to automatically calculate an actual ergonomic risk or load value for the performed work task using the said force-assisting equipment.

Moreover, the invention relates to a system for use when a user performs a work task, wherein the user wears and uses a wearable, actively controlled piece of force-assisting equipment used to assist the user in the performing of the said work task, which force-assisting equipment comprises a force-exerting means for assisting the user in applying a particular force and/or for performing movements of a particular type, and a sensor means for sensing a force applied by the user, wherein an assisting force exerted by said force-exerting means is feedback-controlled based on an instantaneous measurement value from the sensor means, which system is characterised in that the system comprises measuring means arranged to, during the performance of said work task, measure said measurement value using the sensor means for use in said feedback control, and in that the system furthermore comprising calculation means arranged to, using the measurement value, automatically calculate an actual ergonomic risk or load value for the performed work task using the said force-assisting equipment.

Furthermore, the invention relates to a computer software product for use when a user performs a work task, wherein the user wears and uses a wearable, actively controlled piece of force-assisting equipment used to assist the user in the performing of the said work task, which force-assisting equipment comprises a force-exerting means for assisting the user in w applying a particular force and/or for performing movements of a particular type, and a sensor means for sensing a force applied by the user, wherein an assisting force exerted by said force-exerting means is feedback-controlled based on an instantaneous measurement value from the sensor means, which computer software product is characterised in that the computer software product is arranged to perform the steps of a) during the performance of said work task, performing said feedback control loop; b) also during the performance of said work task, receiving said measurement value from the sensor means for use in said feedback control; and c) using the measurement value to automatically calculate an actual ergonomic risk or load value for the performed work task using the said force-assisting equipment.

In the following, the invention will be described in detail, with reference to exemplifying embodiments of the invention and to the enclosed drawings, wherein:

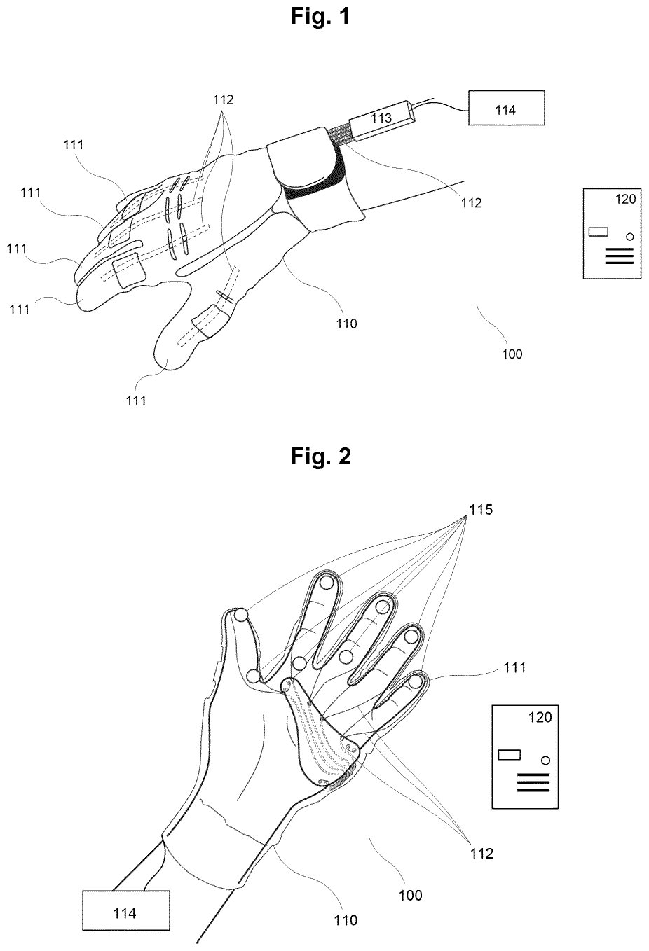

FIGS. 1 and 2 are respective schematic overviews, as seen from two different angles, of a system according to the present invention using a force-assisting glove 110, arranged to perform a method according to the present invention; and

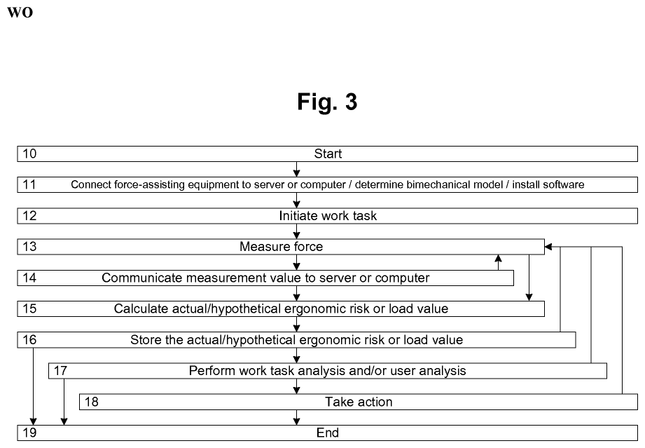

FIG. 3 is a flowchart illustrating a method according to the present invention.

Hence, FIGS. 1 and 2 show a system 100 according to the present invention, for use when a user performs a particular work task. The work task may be any industrially performed work task involving the user in question manually performing work and/or applying a force.

Examples include the mounting of a screw; the lifting or carrying of an article, the operation of a piece of machinery, and so forth.

In general, during the course of the performance of such a work task, the user wears and uses a wearable, actively controlled piece of force-assisting equipment 110 used to assist the user in the performing of the said work task.

Examples of such force-assisting equipment 110 include various types of exoskeleton-type and similar wearable tools, aiding the user in performing particular activities involving the w application of force and/or performing movements. Such forces and/or movements may, for instance, involve only one joint of the user, a particular body part or even substantially the whole muscular controlled body of the user, as the case may be. One particular example is a wearable strengthening glove, such as a glove using artificial tendons running across the fabric of the glove for assisting the user's applied hand and finger force. Another particular example is a similarly wearable shoulder assisting device, using artificial tendons to aid a user raising his or her arm at the shoulder joint.

When the user performs a particular work task using a particular force-assisting equipment having certain force sensors, the methodology of the present invention can be applied to the force-assisting equipment as a whole, taking into consideration measurement values from all force sensors that are relevant to the work task in question performed. It is also possible to use the present invention to evaluate the ergonomic risk or load of only a part of the assisted body part of the user, taken in isolation. For instance, using a force-assisting glove arranged to assist the user in applying force in each of the hand's five fingers, only one or some of the fingers can be continuously evaluated with respect to actual and/or hypothetical risk or load based upon measured forces from sensors only with respect to that or those fingers in question.

That the force-assisting equipment 110 is “actively controlled” means that its operation is arranged for being under active control based upon dynamically changing conditions, such as changing sensed forces, movements, accelerations, angles, postured, and so on. This active control is performed using an active control loop. One particularly interesting example of such an active control loop is a feedback control loop controlling the assisting force instantaneously applied by the equipment 110 in question, taking one or several of the below discussed sensor measurement data as input to the feedback loop.

In FIGS. 1 and 2, as an example of a force-assisting equipment 110 a strengthening glove is shown, comprising glove fingers 111 with artificial tendons 112 driven by an electric or hydraulic motor unit 113 under the control of an electronic control unit 114.

Moreover according to the present invention, the force-assisting equipment 110 in question comprises a force-exerting means for assisting the user in applying a particular force and/or for performing movements of a particular type, as a part of performing the said work task. Such a force-exerting means may, for instance, be the motor 113 arranged to apply a particular force to the artificial tendons 112 in turn arranged to pull at a particular point on the user's body so as to assist the user to apply a force and/or to perform a movement.

Also, the force-assisting equipment 110 comprises a sensor means 115 for sensing an instantaneous force applied by the ergonomically aided user during the performance of the work task in question. Preferably, the sensor means 115 may be arranged to measure such an instantaneous force continuously or repeatedly at least several times per second. The sensor means 115 may be any suitable force sensor unit, and may be connected to the control unit 114.

The sensor means 115 may be arranged to measure a pressing force between a body part of the user and a particular object currently being used or manipulated by the user during the course of the work task performed, such as a pressing force between the inside of the user's finger and a gripped object; or a rotational force applied about a joint of the user's body. The sensor means 115 may furthermore be arranged to measure additional aspects, such as angles or postures of the various body parts of the user.

In some embodiments, the sensor means 115 may also be an indirectly sensing arrangement. For instance, the sensor means may comprise a posture or angle sensor, such as arranged to measure a current angle of extension of an arm of the user in relation to the vertical. Then, using known information about the specific biomechanical system in which the force-assisting equipment is used, such as how the gravitational pull of the arm varies with the measured arm angle, the sensor means can calculate a force value which may then be used by the present invention.

Then, the force-assisting equipment 110 is arranged to control an assisting force exerted by the force-exerting means in the way described above. Such control of the assisting force is preferably performed in real-time, as the user performs the work task in question. For instance, the magnitude of the assisting force may be updated continuously, or at least several times per second, during the performance of the work task. For instance, the force-assisting equipment 110 may be arranged to apply an aiding force which is proportional to a corresponding force applied by the skeletal muscles of the user; or to regulate a predetermined constant force between the user's body and a manipulated object.

According to the invention, the control of the assisting force is a feedback control, performed based on an instantaneous force measurement value from the sensor means 115. For instance, the feedback control may be arranged to, during a particular work task or a particular moment or subpart of a work task, allow the user to apply a predetermined force or to allow the user to apply an artificially amplified force based on a measured force instantaneously applied by the user's own skeletal muscles. In the particular examples mentioned above, the assisting force may be a hand gripping force and an arm lifting force, respectively.

FIGS. 1 and 2 also disclose a computer or server 120, which may be comprised in or connected to the force-assisting equipment 110. The computer or server 120 may be a standalone piece of hardware, a distributed computer functionality, a remotely accessible computer functionality, and so on. In general, the feedback control described above may be performed by a control unit 114 which is actually a part of the force-assisting equipment 110 hardware, physically arranged on or in close proximity to the user's body. However, part or the whole of the feedback control may be performed by the computer or server 120 instead of, or in cooperation with, the control unit 114, as the case may be. In other cases, the functionality performed by the computer or server 120 may be entirely encompassed by the control unit 114.

It is realized that the system 100 according to the present invention preferably comprises both the force-assisting equipment 110 and the computer or server 120. However, the inventive system 100 may also be post-installed on or using an already-existing force-assisting w equipment. Such post-installation may be performed by connecting the force-assisting equipment, wirelessly or using a wire, to the computer or server 120 in turn running computer software arranged to perform the necessary steps of a method according to the present invention. Alternatively, such software may be installed and executed on an existing control unit 114 and/or an existing computer or server 120 to which the force-assisting equipment 110 is already connected. Hence, the system 100 may comprise both the force-assisting equipment 110 and the computer or server 120 including any software arranged to, when executed, perform the method steps included in a method according to the invention. As an alternative, the invention may be implemented as such a software function, which in turn may be installed for execution on or from the existing force-assisting equipment 110 and/or the existing computer or server 120.

FIG. 3 illustrates a method according to the present invention. In a first step 10, the method starts.

In an optional subsequent step 11, the force-assisting equipment 110 is connected to the server or computer 120, alternatively the computer or server 120 is arranged as a part of the force-assisting equipment 110, as described above, for automatic communication of said force measurement data value from said fore-assisting equipment 110 to said server or computer 120. Also in step 11, the said software may be installed, such as on the existing hardware.

In a subsequent step 12, the work task is initiated. As described above, the work task may be any work task during which the work task performing user uses the force-assisting equipment 110 for better performing the work task in question.

In a subsequent step 13, performed during the performance of said work task, the above-discussed force measurement value is measured using the sensor means 115. As also described above, the force measurement value is measured for, and used in, said feedback control performed by the control unit 114 to aid the user in the work task in question the performance of which is ongoing. Such force measurement may hence be performed using the sensors 115 and for instance be performed continuously or intermittently. In general, the force measurement value will be representative of a force presently applied by the user, by aid of the force-assisting device 110, onto an object with which the user currently is working during the course of the said work task. It is understood that the force measurement value may pertain to one or several individual forces. For instance, each of the force sensors 115 may be individually sampled continuously or intermittently. In step 13, a movement and/or posture can also be measured, as described below.

In case the server or computer 120 was connected in step 11, in a subsequent step 14, said force measurement data value or values is or are communicated from the force-assisting equipment 110 to the server or computer 120.

In a subsequent step 15, the said measurement data is used, by the control unit 114, the computer or server 120 and/or the computer software, as discussed above, to automatically calculate an actual, current, ergonomic risk or load value for the performed work task in question, which work task is performed using the same force-assisting equipment 110 which is used to assist the user physically when performing said work task.

It is particularly preferred that the force-assisting equipment 110 is an existing force-assisting equipment which is not modified specifically for being used in the present method. In this case, step 15 may be performed by the said computer software executed on or from said server or computer 120.

In a subsequent step 16, the calculated actual ergonomic risk or load value is stored, in a database or memory accessible by the said computer software, such as in the control unit 114 and/or in the server or computer 120.

Thereafter, in a step 19, the method ends. However, before step 19, the method may reiterate, from step 16, any number of times back to step 13, such as for the duration of at least part or, or even the whole of, the work task in question. Hence, steps 13-15 or 13-16 may be performed repeatedly or continuously during the performance of at least part of the w work task, or even during the whole performance of the work task in question.

Is illustrated in FIG. 3, the method may also reiterate back to step 13 from step 14, in which case several force measurement values may first be recorded, and the actual ergonomic risk or load may be calculated based upon such a batch of force measurement values.

As mentioned, the force value is measured using the same sensors 115 that are used in the force-assisting feedback loop used to assist the user in the work task. Since the system 100 has up-to-date knowledge of the assisting force being applied, such as via tendons 112, the system 100 can calculate the force actually currently imparted by the skeletal muscles of the user, for instance as a simple subtraction from a measured contact force of the applied assisting force transformed to a corresponding applied contact force at the sensor 115 in question.

Since the system 100 also has knowledge about which sensor 115 provides which force measurement value, it is possible to relatively easily achieve a detailed view of the forces currently being applied by the user's skeletal muscles, and hence the ergonomic situation of the user at each respective moment in time. This may even be achievable without a specific calibration step.

In order to achieve such a detailed view of the currently imparted forces of the user, it is preferred that at least 3, preferably at least 5, force sensors are used simultaneously by the system 100 for performing such force measurements continuously or intermittently. Furthermore, it is preferred that a respective assisting force is applied using at least three individually and simultaneously operating force assisting means, such as the tendons 112 that are individually pulled by the motor 113. In order to calculate a current ergonomic status of the user, the force measurement values and/or the assisting forces may be transformed into a common geometry in which they can be compared directly one to the other. Such transforms may be part of the physical ergonomic model discussed below.

It is understood that, in the system aspect of the present invention, the system 100 comprises force measuring means arranged to, during the performance of said work task, perform the said force measurement using the sensor means 115 for use in said feedback control. Hence, the measuring means may, for instance, comprise the sensors 115 and the control means 114 arranged to receive the read sensor data from the sensors 115. Moreover, in this case the system 100 furthermore comprises calculation means, such as the above discussed computer software, which calculation means is arranged to, using the said force measurement data, automatically calculate said actual ergonomic risk or load value for the performed work task using the force-assisting equipment 110. In general, the system 100 may comprise the software described herein as a subpart.

Using such a method and such a system 100, it is hence possible to achieve an accurate, up-to-date and real-time estimation of an actual ergonomic risk or load for the user actually and currently performing the work task in question. Such ergonomic risk or load value may be used in a variety of different ways, as will be further detailed below and in the following.

For instance, when applied to a group of users performing one and the same work task, users having a relatively high actual ergonomic risk or load can be automatically identified, indicating potential maluse of a tool or similar; or users not being properly trained for that work task may be automatically identified.

The calculated actual ergonomic risk or load can also be monitored over time and used as an input for automated work task rotation. Preferably, the said computer software is connected to a work planning database, and comprises algorithms for automatically adapting a work task rotation plan as a result of an unexpectedly high calculated actual ergonomic risk or load value for at least one user. For instance, the value may be regarded as unexpectedly high if the corresponding actual ergonomic risk or load value for the user in question and for the same or corresponding work task jumps more than a predetermined amount or percentage over a particular maximum or predetermined time interval. In this case, the user in question may be assigned to a less ergonomically straining work task so as to avoid overwork for that particular user. In other embodiments, a work task rotation plan can be completely automatically proposed by the said computer software based upon recently calculated actual ergonomic risk or load values, for instance by minimizing a risk for overwork for individual identified users.

The calculated actual ergonomic risk or load values may also be used to evaluate and iteratively adapt changes in a production process involving manual work by individual users, such as the introduction of new work flows or tools. By quickly identifying possibly risky work tasks, the production process may be automatically and preferably immediately adjusted so as to avoid overworked users. The said computer software is preferably arranged to continuously monitor the calculated actual ergonomic risk or load values and set off an alert in case a particular work task is identified as producing higher actual ergonomic risk or load values than a predetermined threshold value, either on average across several users or for more than a predetermined number of individual users.

Hence, the present invention can be used as a way to improve the long-term and short-term health of users in a particular production flow, and generally results in lower costs for healthcare.

It is understood that, in order to be able to perform the above tasks, the computer software preferably has access to, such as via an updated database of the above mentioned type, with information regarding what work task each user is currently performing, and what individual user is currently using the force-assisting equipment 110.

Additional benefits of the present invention relate to production process improvements. For instance, particularly low-risk or low-load work tasks can be automatically identified by the computer software, and this information can be used by the computer software to automatically propose or implement an increase of production speed for such identified work tasks. The opposite is true regarding particularly high-risk or high-load work tasks, the speed of which may be automatically decreased. By mapping a set of work tasks in a particular w production flow, ergonomic risks can be identified and handled by automatically adapting the production planning. For instance, identified particularly high-risk or high-load work tasks can be distributed to a larger subset of the working users, so as to avoid overworked users.

Furthermore, the invention can be used during education of new users. For instance, a user under education can perform a particular work task and automatically and preferably immediately receive feedback regarding the calculated actual ergonomic risk or load value resulting from the work. Then, the user may implement iterative adjustments in his or her technique so as to improve the ergonomics while performing the work task.

Moreover, the production quality may be improved using the present invention by reducing user malpractice due to fatigue. This is achieved not only as a consequence of avoiding overworked users, but also by decreasing fatigue from an acceptable level to an even lower level. All this can be performed automatically, and without adding any manual steps.

It is noted that the said computer software may preferably be arranged with algorithms for automatically performing these method steps, and to either produce a suggestion for planning adjustments to an operator, or automatically implement such adjustments in a production planning which is automatically supplied by the computer software.

The present invention can also be used for workplace certification purposes; for decreasing the need for subjective judgements regarding ergonomics; and for general workplace ergonomics mapping purposes.

It is noted that the computer software, using the actual ergonomic risk or load values calculated for a set of work tasks and for a set of users, for all the scenarios described above, can be used to assess the ergonomic status of both particular work tasks (independently of user) and for particular identified users (independently of work task). Then, combinations between these two estimations may be used for additional purposes. For instance, the ergonomic strain for a particular user performing a particular work task can be automatically assessed based upon a “normal” ergonomic strain for an average or typical user performing the work task in question.

Analyses and actions of the above discussed types are performed in an optional step 17. Then, the method may also reiterate back to step 13 from step 17, as the case may be.

In some embodiments, in addition to or instead of the said actual ergonomic risk or load value, in step 15 a hypothetical ergonomic risk or load value is also calculated for the performed task. This hypothetical ergonomic risk or load value is calculated based upon the same force measurement value as the corresponding actual ergonomic risk or load, and so as to reflect the hypothetical situation in which the work task in question was performed without the use of the force-assisting equipment 110. In other words—if the user performed the work task without using the force-assisting equipment 110, what would the ergonomic risk or load be then?

Thus, the hypothetical ergonomic risk or load value is calculated based upon to what extent the user is assisted by the force-assisting equipment 110, in turn determined by the currently measured force using which the force-assisting equipment 110 aids the user in performing the work task in question. For instance, in a simple exemplifying case the hypothetical ergonomic risk or load value may be calculated based upon the actual measured force applied by the user to the workpiece, as measured by the sensors 115, irrespective of any aiding force applied by the force-assisting equipment 110 with the purpose of aiding the user in performing the work task in question.

Then, the calculated actual ergonomic risk or load value is stored, in step 16, possibly together with the calculated hypothetical ergonomic risk or load value as described above.

The hypothetical ergonomic risk or load value may be used in an automatic production process planning algorithm performed by the said computer software, in which it is assessed what work tasks require the use of force-assisting equipment 110; what categories of users w should be assigned to what work tasks; what work task rotation schemes to apply; and so on.

In some embodiments, the instantaneous hypothetical ergonomic risk or load value, or the hypothetical ergonomic risk or load value measured over time, may be used in combination with the corresponding actual ergonomic risk or load value measured instantaneously or over time, in order to further improve the quality of a production process. For instance, by considering the instantaneous or time-integrated hypothetical ergonomic risk, harmful work tasks can be identified, such as by allowing workers to perform a number of potentially harmful tasks while performing ergonomic risk measurements as described herein. For each identified such harmful work task, the instantaneous or time-integrated actual ergonomic risk, as measured from the same test runs, can be used to determine whether or not the use of force-assisting equipment is enough to reach ergonomic goals, or if the work task needs to be redesigned so as to be less ergonomically challenging.

Measured hypothetical and/or actual ergonomic risks or loads may also be used in a feedback loop for control of the force-assisting equipment itself. For instance, a currently measured instantaneous and/or time-integrated actual ergonomic risk or load value can be used as an input value or trigger for a modification of a control program used by the force-assisting equipment used to perform the same work task in relation to which the said ergonomic risk or load value is currently being measured. Such feedback is hence performed in realtime or near realtime. For instance, if an actual ergonomic risk which is higher than an allowed threshold value is detected, the control program can be adjusted so as to provide stronger force-assisting action by the force-assisting equipment. For instance, the force-assisting equipment may, in its own force-assisting feedback loop program, use a parameter providing a certain force-assist as a percentage of a corresponding force currently applied by the user, in which case this parameter is modified upwards as a result of the detected unacceptably high ergonomic risk. Another example is when the said control program implements a maximum force-assist value, which maximum force-assist value may be increased as a result of the detected unacceptably high ergonomic risk.

As understood from the above, the measurement step 13 may be performed instantaneously, at a particular point in time. Furthermore, the calculation step 15 may be performed based on a plurality of different such instantaneous measurement values from a certain time period during the performance of the work task.

According to a very preferred embodiment, the method further comprises an initial step, such as a part of step 11, in which a physical ergonomic model, which is specific to the particular work task or to the particular piece of force-assisting equipment 110, or preferably specific to both these aspects, is determined. The physical ergonomic model is further determined based upon physical characteristics of the work task in question, as well as physical characteristics of the sensor means 115, respectively, as the case may be. It is important that the physical ergonomic model is fully determined before step 15, in the sense that the calculation(s) performed in step 15 can be fully automated and not require any manual input from an operator. In particular, it is preferred that the physical ergonomic model determined in step 11 is used as a basis for the calculation(s) in step 15.

According to the physical ergonomic model, the said force measurement data is used as an input to the physical ergonomic model, and the actual and/or hypothetical ergonomic risk or load value is calculated as an output based on the said input force measurement data. Hence, the above mentioned geometric transforms may be a part of the physical ergonomic model. As an additional example, the physical ergonomic model may take into numerical consideration the fact that certain types of movements may be associated with different types of relationships between applied force, movement and ergonomic risk or load.

In general, the actual and/or hypothetical ergonomic risk or load may be calculated in step 15 based upon the measured force data from the sensors 115. However, it is understood that the sensors 115 may also comprise movement and/or posture sensors, the measurement values of which may also be input, such as via the control unit 114, to the physical ergonomic model and used to calculate the actual and/or hypothetical ergonomic risk or load. Hence, the currently applied force may result in different ergonomic risks or loads w depending on a motion pattern currently being pursued by the user and/or a current posture of the relevant body part of the user.

In a particular embodiment, the said physical ergonomic model comprises an implementation of a HAL-TLV (Hand Activity Level Threshold Limit Value) and/or KIM (Key Indicator Method) evaluation model, where the input data of the respective evaluation model are completely provided in an automatic way based upon the read sensor 115 data as described above. These evaluation models have been proven to yield relevant values, but have not to date been useful in fully automated applications.

In an exemplifying example, incorporated herein in order to improve the understanding of how to implement the present invention in practise, it was assessed how much ergonomic risk a particular person was subjected to when doing three different grasp intensive work tasks while using a force-assisting equipment in the form of a force-assisting glove. Both the hypothetical risk level and the actual risk level were assessed for these work tasks. A HAL-TLV was used as ergonomic evaluation model and tailored to the particular force-assisting glove used. The HAL-TLV model used has two risk levels that are special, an action level (AL) threshold value=0.56 and a threshold limit value (TLV)=0.78. When a risk level calculated based upon measurements is below the AL value, the ergonomic risk is considered low, and no action is required. When the calculated risk is between AL and TLV, a redesign of the work place should be considered. If the risk is above TLV, a redesign of the work place is considered necessary.

When the ergonomic risk level had been evaluated, a particular action based on the HAL-TLV ergonomic evaluation model was proposed. In this example, this was made manually. However, it is realized that such proposing can be made automatically as described herein.

The actual risk when using the force-assisting equipment is lower than the hypothetical risk level, since the force-assisting equipment applies force when the user grasps objects, thereby reducing the force the user needs to apply to do the work task in question.

As mentioned above, three different tests were performed according to the following.
A paving stone weighing 3 kg was lifted a short distance and then put back down. This test simulates a work task where paving stones are placed next to each other.
Buttons were pinched together in quick succession using the user's thumb and the index finger. This is to simulate pinching tasks.
A screwdriver was used to fasten screws. It was actuated with the user's index finger as trigger. This simulates work tasks such as fastening screws ore bolts.

For each test, the grasping action was repeated 50 times and an average was calculated.

The ergonomic risk according to the HAL TLV evaluation model was calculated as follows:

$${HAL}_{{TLV}_{SCORE}} = \frac{FORCE}{FORC {E}_{MAX}} * \frac{10}{10 - \frac{6.56 ln ( DUTY CYCLE ) FREQ U E N C {Y}^{1.31}}{1 + 3.18 FREQUENC {Y}^{1.31}}} ,$$

Where FORCE_{MAX} is a norm value which is determined based upon the user's maximum strength. Such calculations will be well-known for the person skilled in the relevant arts.

The following table summarizes the results of these three tests:

| | | Actual risk (with | | |
|---|---|---|---|---|
| | | force-assisting | | |
| Test | Hypothetical risk | equipment(FAE)) | Analysis | Action |
| Paving | 0.82 | 0.36 | 0.82 > TLV → | Use the FAE to |
| stone | Force = 15.2 N | Force = 6.6 N | High risk. | reduce the risk |
| | Duty cycle = 40% | Duty cycle = 40% | 0.36 < AL → Low | level to an |
| | Frequency = 0.25 Hz | Frequency = 0.25 Hz | risk | acceptable level |
| | | | This work task | |
| | | | should not be | |
| | | | performed with- | |
| | | | out the FAE | |
| Button | 0.91 | 0.51 | 0.82 > TLV → | Use the FAE to |
| pinch | Force = 10 N | Force = 5.6 N | High risk. | reduce the risk |
| | Duty cycle = 36% | Duty cycle = 36% | 0.51 < AL → Low | level to an a |
| | Frequency = 1.0 Hz | Frequency = 1.0 Hz | risk | acceptable level |
| | | | This work task | |
| | | | should not be | |
| | | | performed with- | |
| | | | out the FAE | |
| Screwdriver | 0.59 | 0.28 | AL < 0.59 < TLV | Be cautious. If |
| | Force = 12 N | Force = 5.7 N | → Medium risk, | the risk in- |
| | Duty cycle = 60% | Duty cycle = 60% | This work task | creases consider |
| | Frequency = | Frequency = 0.15 Hz | can be per- | using an |
| | 0.15 Hz | | formed without | FAE. |
| | | | obvious risks but | |
| | | | a lower risk is | |
| | | | preferred | |

In a preferred embodiment, the force-assisting equipment is arranged specifically for assisting movements and/or force of the user's shoulder, arm, hand, leg and/or torso.

In a preferred embodiment, the method comprises as a part of step 17 an analysis and decision step, in which the calculated actual and/or hypothetical ergonomic risk or load value is used for taking a decision affecting how, if, for how long, or when the work task is performed.

In particular, the said decision step may comprise a comparison between firstly the calculated actual and/or hypothetical ergonomic risk or load value, in the form of an instantaneous ergonomic risk or load value or an integrated total ergonomic risk or load value, and secondly a predetermined corresponding threshold value. If the calculated actual and/or hypothetical ergonomic risk or load value is found to be higher than said threshold value, the method is arranged to achieve an alarm signal.

Herein, the term “alarm signal” should be determined broadly. Hence, apart from an audiovisual, physical alarm signal, the signal achieved may be a digital or electric signal signifying a particular state into which the system 100 enters as a result of the alarm signal. Hence, the system 100 may be put into a warning state or similar, such as with respect to the work task and the user in question for which the ergonomic situation was measured and evaluated. This state change of the system 100 may then, in turn, result in any of a number of possible outcomes, performed in an optional action taking step 18, performed after step 17.

For instance, in one exemplifying embodiment step 18 may comprise causing the force-assisting equipment 110 to limit the user's movements as a result of the detection of said alarm signal. Such limitation may be achieved in any suitable way, and may be of a forcing type or merely informing the user of the suitability of no longer performing the work task under the current conditions.

For instance, said limitation imparted in step 18 may be achieved by the force-assisting equipment 110 denying the user sufficient force assisting to be able to perform certain predetermined movements and/or movements requiring the user to use a force exceeding a force threshold value, or that the user is alerted regarding the detected alarm signal. This may be achieved by the said computer software, such as by the force-assisting equipment 110 itself and/or the computer or server 120. The information in question may be presented to the user via a suitable interface, such as a graphical display or warning lamp on the force-assisting equipment 110 and/or the computer or server 120.

Above, preferred embodiments have been described. However, it is apparent to the skilled person that many modifications can be made to the disclosed embodiments without departing from the basic idea of the invention.

For instance, as has been mentioned in a number of contexts above, the force-assisting equipment 110 may be arranged to assist the user in the imparting of a static and/or dynamic force, such as when using a screwdriver or pressing two objects together or apart. The force-assisting equipment 110 may in addition, or instead, be arranged to assist the user in the performing of various movements. All which has been said above relates equally w well to both these cases, as applicable. In a more general sense, the force-assisting equipment 110 may be labelled as a “strengthening” equipment.

Furthermore, it is realized that the force-assisting equipment 110 may have any other properties in addition to the ones described above.

It is understood that all which has been said in relation to the present method is equally applicable to the present system 100, and vice versa.

Hence, the invention is not limited to the described embodiments, but can be varied within the scope of the enclosed claims.

1. A method when a user performs a work task, wherein the user wears and uses a wearable, actively controlled piece of force-assisting equipment used to assist the user in the performing of the said work task, which force-assisting equipment comprises a force-exerting means for assisting the user in applying a particular force and/or for performing movements of a particular type, and a sensor means for sensing a force applied by the user, wherein an assisting force exerted by said force-exerting means is feedback-controlled based on an instantaneous measurement value from the sensor means, wherein the method comprises the steps of
a) determine a physical ergonomic model, which is specific to said work task and/or to said piece of force-assisting equipment, based upon physical characteristics of the work task and/or physical characteristics of the sensor means, which model further comprises a transform from an assisting force applied by said force-assisting equipment to a corresponding force imparted by the user's skeletal muscles;
b) during the performance of said work task, measuring said measurement value using the sensor means for use in said feedback control; and
c) using the measurement value and said physical ergonomic model to automatically calculate an actual ergonomic risk or load value, due to forces being applied by the user's skeletal muscles, for the performed work task using the said-force-assisting equipment.

2. The method according to claim 1, wherein the method further comprises, before step a, connecting the force-assisting equipment to a server or computer, alternatively arranging a server of computer as a part of the force-assisting equipment, for automatic communication of said measurement value to said server or computer, and, before step b, communicating said measurement value from the force-assisting equipment to the server or computer.

3. The method according to claim 2, wherein the force-assisting equipment is an existing force-assisting equipment which is not modified specifically for being used in the method, and wherein step b is performed by computer software executed on or from said server or computer.

4. The method according to claim 1, wherein, in addition to the actual ergonomic risk or load value, a hypothetical ergonomic risk or load value is also calculated for the performed task reflecting the hypothetical case in which the force-assisting equipment is not used, which hypothetical ergonomic risk or load value is calculated based upon to what extent the user is assisted by the force-assisting equipment, and wherein the calculated actual ergonomic risk or load value is stored together with the calculated hypothetical ergonomic risk or load value.

5. The method according to claim 1, wherein steps a and b are performed repeatedly or continuously during the performance of at least part of the work task.

6. The method according to claim 5, wherein the calculation in step b is based on a plurality of different instantaneous measurement values from a certain time period during the work task.

7. The method according to claim 1, wherein the actual ergonomic risk or load value according to the physical ergonomic model is calculated as an output based on the measurement value.

8. The method according to claim 7, wherein the physical ergonomic model comprises a HAL and/or KIM evaluation model.

9. The method according to claim 1, wherein the force-assisting equipment is arranged specifically for assisting force of the user's shoulder, arm, hand, leg and/or torso.

10. The method according to claim 1, wherein the method comprises a decision step in which the calculated actual ergonomic risk or load value, and possibly also a calculated hypothetical ergonomic risk or load value, is used for taking a decision affecting how, if, for how long, or when the work task is performed.

11. The method according to claim 10, wherein the decision step comprises a comparison between firstly the calculated actual ergonomic risk or load value, in the form of an instantaneous risk or load value or an integrated total risk or load value, and secondly a predetermined corresponding threshold value, and if the calculated actual ergonomic risk or load value is found to be higher than said threshold value to achieve an alarm signal.

12. The method according to claim 11, wherein the method comprises causing the force-assisting equipment to limit the user's movements as a result of the detection of said alarm signal.

13. The method according to claim 12, wherein said limitation is achieved by the force-assisting equipment denying the user sufficient force assisting to be able to perform a predetermined work task requiring the user to use a force exceeding a force threshold value, or that the user is alerted regarding the detected alarm signal.

14. A system for use when a user performs a work task, wherein the user wears and uses a wearable, actively controlled piece of force-assisting equipment used to assist the user in the performing of the work task, which force-assisting equipment comprises a force-exerting means for assisting the user in applying a particular force and/or for performing movements of a particular type, and a sensor means for sensing a force applied by the user, wherein an assisting force exerted by said force-exerting means is feedback-controlled based on an instantaneous measurement value from the sensor means, wherein the system comprises measuring means arranged to, during the performance of said work task, measure said measurement value using the sensor means for use in said feedback control, wherein the system furthermore comprising calculation means arranged to, using the measurement value, automatically calculate an actual ergonomic risk or load value, due to forces being applied by the user's skeletal muscles, for the performed work task using the force-assisting equipment, and wherein the calculation means is further arranged to perform said automatic calculation using a determined physical ergonomic model, which model is specific to said work task and/or to said piece of force-assisting equipment, which model is determined based upon physical characteristics of the work task and/or physical characteristics of the sensor means, and which model further comprises a transform from an assisting force applied by said force-assisting equipment to a corresponding force imparted by the user's skeletal muscles.

15. A non-transitory computer readable medium storing a computer software product for use when a user performs a work task, wherein the user wears and uses a wearable, actively controlled piece of force-assisting equipment used to assist the user in the performing of the work task, which force-assisting equipment comprises a force-exerting means for assisting the user in applying a particular force and/or for performing movements of a particular type, and a sensor means for sensing a force applied by the user, wherein an assisting force exerted by said force-exerting means is feedback-controlled based on an instantaneous measurement value from the sensor means, wherein the computer software product, when executed, is arranged to perform the steps of
a) during the performance of said work task, performing said feedback control loop;
b) also during the performance of said work task, receiving said measurement value from the sensor means for use in said feedback control; and
c) using the measurement value to automatically calculate an actual ergonomic risk or load value, due to forces being applied by the user's skeletal muscles, for the performed work task using the force-assisting equipment,
and wherein the automatic calculation in step c is performed using a determined physical ergonomic model, which model is specific to said work task and/or to said piece of force-assisting equipment, which model is determined based upon physical characteristics of the work task and/or physical characteristics of the sensor means, and which model further comprises a transform from an assisting force applied by said force-assisting equipment to a corresponding force imparted by the user's skeletal muscles.
